(19) United States
(12) Patent Application Publication
(10) Pub. No.: US 2026/0098854 A1
(43) Pub. Date: Apr. 9, 2026

(54) MICROPILLAR CONSTRUCTS FOR MEASURING CONTRACTILE FORCE OF ANNULAR TISSUES

(71) Applicant: University of Virginia Patent Foundation, Charlottesville, VA (US)

(72) Inventors: Shayn Peirce-Cottler, Charlottesville, VA (US); Dorothy N. Beck, Charlottesville, VA (US); Emily Ferrarese, Charlottesville, VA (US); Bette Dzamba, Charlottesville, VA (US); David R. Shook, Charlottesville, VA (US); Christopher B. Highley, Crozet, VA (US); Douglas W. DeSimone, Charlottesville, VA (US); Tien Comlekoglu, Charlottesville, VA (US)

(21) Appl. No.: 19/346,879

(22) Filed: Oct. 1, 2025

Related U.S. Application Data
(60) Provisional application No. 63/703,819, filed on Oct. 4, 2024.

(51) Int. Cl. G01N 33/483; B29C 64/129; B29K 71/00; B29K 105/00; B29L 31/00; B33Y 10/00; B33Y 70/10; B33Y 80/00

(52) U.S. Cl. CPC G01N 33/4833; B29C 64/129; B33Y 10/00; B33Y 70/10; B33Y 80/00; B29K 2071/00; B29K 2105/0005; B29L 2031/753

(57) ABSTRACT

The present disclosure provides for micropillar constructs for measuring contractile force of annular tissues, methods of quantifying biomechanical forces of a tissue, systems for quantifying biomechanical forces of a tissue, and the like.

### CROSS-REFERENCE TO RELATED APPLICATIONS

This application claims the benefit of U.S. Provisional Application entitled “MICROPILLAR CONSTRUCTS FOR MEASURING CONTRACTILE FORCE OF ANNULAR TISSUES” and having Ser. No. 63/703,819, filed Oct. 4, 2024, which is herein incorporated by reference in its entirety.

### STATEMENT OF GOVERNMENT INTEREST

This invention was made with government support under GM147410, GM140008, GM131865, and HL155143 awarded by the National Institutes of Health, and 2140549 awarded by the National Science Foundation. The government has certain rights in the invention.

### BACKGROUND

Understanding the mechanical forces generated by cells and tissues of complex three-dimensional geometries on their substrates and environment can be important for investigating many biological processes such as tissue morphogenesis, wound healing, tumorigenesis, and collective cell migration. Current methods for quantifying forces generated by tissues on their environment such as Traction Force Microscopy (TFM) support mainly planar tissue geometries. These methods often are insufficient for investigating the contractile forces generated by complex three-dimensional tissues and tissue explants because they measure interactions of tissues resting on a substrate and are unable to measure forces generated by cells and tissues resting above the substrate. In addition, current methods often do not support tissues at a micrometer scale or can be too stiff to be sensitive to weaker forces on the microscale.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1C illustrate manufactured hydrogel micropillars for inference of toroidal tissue contractile forces. FIG. 1A shows a technical drawing of the micropillar construct design according to various embodiments of the present disclosure. Large blocks of base material elevate contractile tissue to increase bending moment on the pillars and help ensure DLP print fidelity. FIG. 1B shows stereo zoom imaging of the printed micropillars threaded through a contractile toroidal tissue explant dissected from the developing Xenopus laevis embryo to infer forces generated by the tissue according to various embodiments of the present disclosure. Arrows indicate the direction of tissue contractility. FIG. 1C shows stereo zoom imaging of the pillar construct according to various embodiments of the present disclosure. The red fluorescence is the result of thiolated fluorophore conjugated to the pillar material, demonstrating potential as a flexible patterned microenvironment.

FIG. 2 shows a hydrogel stress-strain relationship determined through dynamic mechanical analysis. The stress and strain response under both compressive and tensile loading is shown for a selected bioink formulation. Data shown represents the mean of n=3 replicates in compression, n=2 replicates in tension prior to material failure according to various embodiments of the present disclosure.

FIGS. 3A-3C show micromechanical testing of printed structures reveals a linear force displacement relationship. FIG. 3A shows applied force and resulting pillar deflection measures during cyclic micromechanical testing for 4 experimental replicates with sample large prints approximately 150 μm in semicircular cross section radius. FIG. 3B shows loading and unloading data during micromechanical testing reveal a linear force-displacement relationship from which the relative contractile force can be inferred. FIG. 3C shows a representative image of micromechanical testing setup according to various embodiments of the present disclosure. Measurement probe deflects the pillars horizontally and applied force and resulting displacement measurements over time are recorded.

FIG. 4 shows an example micropillar fabrication and force inference process. The prepared bioink is pipetted into a standard 6-well glass bottom plate and placed into a CELLINK BionovaX DLP Printer. The DLP printer prints the specified design. Printed designs can then be used in experiment, and observed deflections can be reproduced with a CellScale MicroSquisher micromechanical testing device to directly measure the force required to displace pillar structures to the degree observed in experiment.

FIG. 5 shows representative micropillar prints. FIG. 5A shows a lateral view of a printed pillar pair for use in biomechanical force measurement. FIG. 5B shows stereo zoom imaging of the pillar construct, red fluorescence is the result of thiolated fluorophore conjugated to the pillar material, demonstrating potential as a flexible patterned microenvironment.

FIG. 6 shows pillar deflections for various pillar diameters in experimental application. 2 mm tall pillars with an intended pillar semicircular cross-sectional radius of 70 μm were tested with contractile toroidal explants.

FIGS. 7A-7O show pillar designs according to various embodiments of the present disclosure. FIG. 7A shows single solid pillar extending from a planar surface, such as the bottom of a well plate. FIG. 7B shows a single hollow pillar extending from a planar surface, such as the bottom of a well plate. FIG. 7C shows a pillar structure including two parallel cylindrical structures extending from a planar surface, such as the bottom of a well plate, and a third cylindrical structure extending perpendicularly from the top of one parallel cylindrical structure to the top of the second cylindrical structure. FIG. 7D shows two solid pillars placed parallel to each other and extending from a planar surface, such as the bottom of a well plate. FIG. 7E shows a plurality of solid pillars extending perpendicularly between a plurality of blocks. Each of the plurality of solid pillars can be placed in parallel with another solid pillar and each pair of pillars can be placed a distance away from each of the plurality of blocks. The pillars can be semicylindrical according to various embodiments of the present disclosure. FIG. 7F shows another plurality of solid pillars extending perpendicularly between a plurality of plates. Each of the plurality of solid pillars can be placed in parallel with another solid pillar and each pair of pillars can be placed a distance away from each of the plurality of plates. The pillars can be semicylindrical according to various embodiments of the present disclosure. FIG. 7G shows a variety of different pillar structure designs according to various embodiments of the present disclosure. FIG. 7H shows a plurality of pairs of semicylindrical pillars according to various embodiments of the present disclosure. FIG. 7I shows a plurality of solid cylindrical pillars in groups of three according to various embodiments of the present disclosure. FIG. 7J shows a plurality of hollow cylindrical pillars according to various embodiments of the present disclosure. FIG. 7K shows a well having a plurality of pillar structures extending perpendicularly from the bottom surface of the well. FIG. 7L shows a plurality of pairs of hollow semicylindrical pillars according to various embodiments of the present disclosure. FIG. 7M shows a plurality of pillars extending perpendicularly from a surface of an “L” shaped block according to various embodiments of the present disclosure. FIG. 7N shows a well having a plurality of holes extending perpendicularly from the bottom surface of the well. FIG. 7O shows a plurality of solid cylindrical pillars extending perpendicularly from a planar surface according to various embodiments of the present disclosure.

### DETAILED DESCRIPTION

The present disclosure is directed to micropillar constructs for measuring contractile force of annular tissues. Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law, allowing for the presence of more than that which is recited so long as basic or novel characteristics of that which is recited is not changed by the presence of more than that which is recited, but excludes prior art embodiments.

### Definitions

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art.

As used herein, a “micropillar” can refer to a microscale columnar structure that can be fabricated from various materials, such as one or more polymers. A micropillar can have a height, a diameter or cross-sectional width, and spacing from adjacent structures (e.g., one or more blocks) that are each on the micrometer scale. A micropillar can have various cross-sectional geometries including circular, square, hexagonal, or custom-designed profiles, and can be fabricated individually or as an array. Fabrication of a micropillar can involve techniques including light-based 3D printing. A micropillar can be used in various embodiments of the present disclosure to quantify one or more forces of a tissue.

As used herein, a “tissue” can refer to an organized assembly of biologically derived or synthetic cells and/or extracellular matrix components that function collectively to perform specific structural or physiological roles within an organism or engineered system. A tissue can be derived from natural biological sources (e.g., mammalian, avian, plant, etc.), cultured in vitro, or fabricated using tissue engineering techniques. A tissue can include one or more cell types embedded in or supported by a scaffold or substrate, and can exhibit characteristics such as vascularization, cellular differentiation, and biochemical functionality. A tissue can include epithelial tissue, connective tissue, muscle tissue, nervous tissue, or combinations of any thereof.

As used herein, a “polymer” can refer to a substance composed of macromolecules formed by the chemical bonding of repeating structural units, or monomers. Macromolecules can be characterized by a high molecular weight and can exhibit linear, branched, crosslinked, or networked architectures. A polymer can be naturally occurring (e.g., cellulose, proteins, gelatin, collagen, chitosan, silk fibroin, hyaluronic acid, DNA, etc.) or synthetically produced (e.g., polyethylene glycol (PEG), methocellulose, 1,3-diglycerolate, polyvinyl alcohol, an acrylate, a methacrylate, a norbornene, a thiol, a vinyl sulfone, a maleimide, etc.) and can be engineered to possess specific physical, chemical, mechanical, thermal, or biological properties. A polymer can be thermoplastic or thermosetting. In various embodiments of the present disclosure, polymers can be used to fabricate micropillars.

As used herein, a “hydrogel” can refer to a three-dimensional, hydrophilic polymer network capable of absorbing and retaining an amount of water or biological fluids while maintaining its structural integrity. A hydrogel can be formed from natural polymers (e.g., alginate, collagen, gelatin, hyaluronic acid, etc.) or synthetic polymers (e.g., polyethylene glycol (PEG), polyacrylamide, polyvinyl alcohol, etc.). Crosslinking of the polymer chains can occur either through physical interactions (e.g., hydrogen bonding, ionic interactions) or chemical bonds (e.g., covalent crosslinking). A hydrogel can be designed to be mechanically tunable, making them suitable for applications including fabrication of micropillars as described herein.

As used herein, a “bioink” can refer to a formulation composed of living cells and/or biologically active components suspended within a biocompatible material matrix that is suitable for use in various 3D bioprinting techniques. A bioink can be designed to support cell viability, proliferation, and function during and after the printing process, and can include hydrogels, extracellular matrix (ECM) components, growth factors, crosslinking agents, or other bioactive molecules.

As used herein, a “light-based 3D printer” or “light-based 3D printing” can refer to an additive manufacturing technique in which patterned light is used to selectively solidify or crosslink a photosensitive material (e.g., a photopolymer resin, a hydrogel, etc.) into a 3D structure. The process can rely on photopolymerization, wherein exposure to specific wavelengths of light (e.g., ultraviolet (UV), visible, or near-infrared) initiates a chemical reaction in the material, resulting in localized curing or solidification. Light-based 3D printing methods can include stereolithography (SLA), digital light processing (DLP), two-photon polymerization (2PP), and volumetric printing.

As used herein, a “substrate” can refer to a base material or surface upon which processes, structures, or layers can be applied, built, or supported. A substrate can serve as a mechanical support or a functional surface and can be composed of a variety of materials including glass, silicon, metal, ceramic, plastic, or polymeric films. A substrate can provide a foundational layer upon which micropillars are fabricated using various light-based 3D printing techniques according to various embodiments of the present disclosure. In various aspects of the present disclosure, a substrate can include a functionalized glass plate or coverslip.

Further definitions are provided in context below. Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

### Discussion

The present disclosure provides for micropillar constructs for measuring contractile force of annular tissues. Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

As compared to current methods for quantifying forces generated by tissues, various embodiments of the present disclosure can be used to infer weak biological forces generated by contractile toroidal-shaped tissue explants through the fabrication of a micropillar construct. In addition, micropillars of the present disclosure can be conjugated with proteins or other active biomimetic peptides to investigate or modulate the force-generating properties of complex biological tissues, such as natively contractile annular-shaped tissues described herein. Various embodiments of the present disclosure can be used to infer the magnitude of forces generated during surface-tension mediated contractility of an explanted tissue.

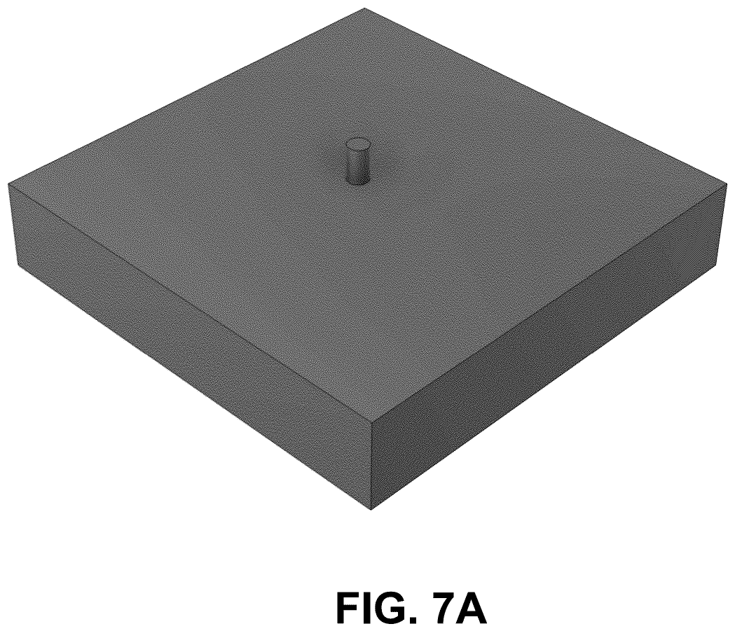

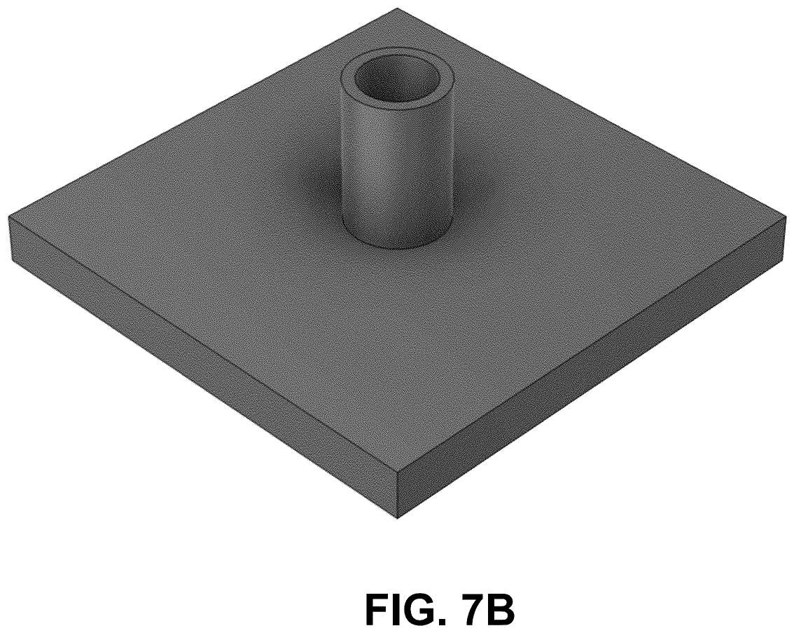

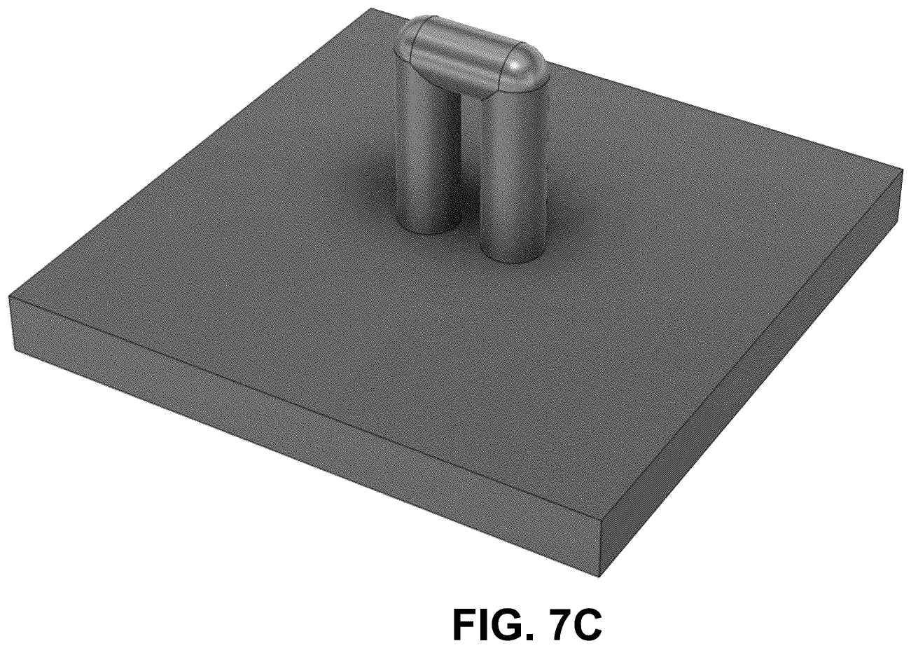

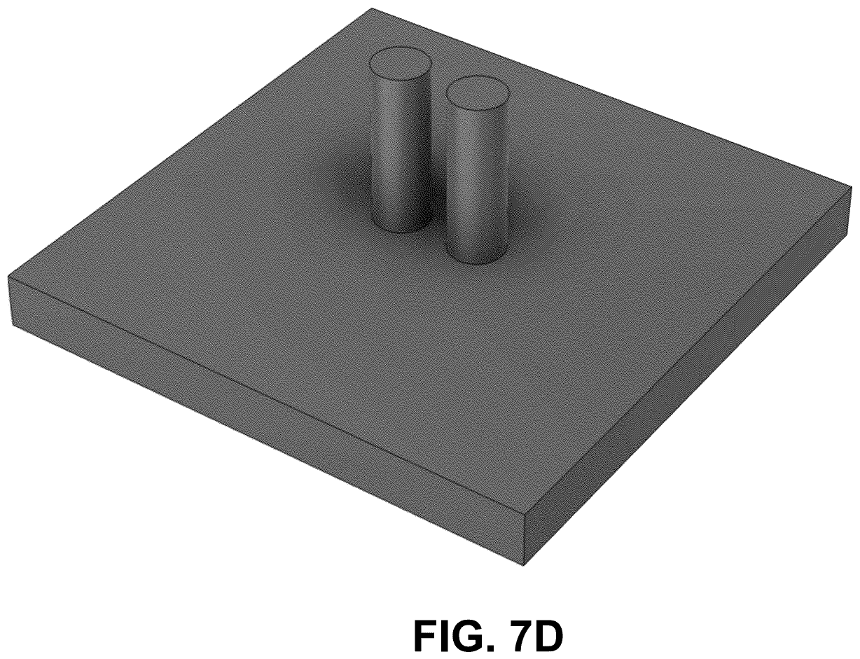

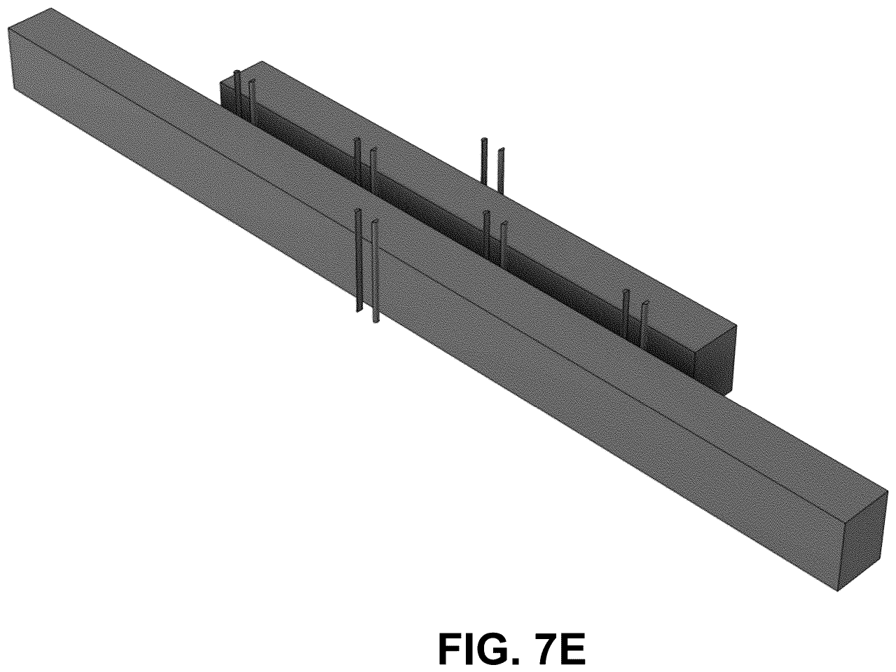

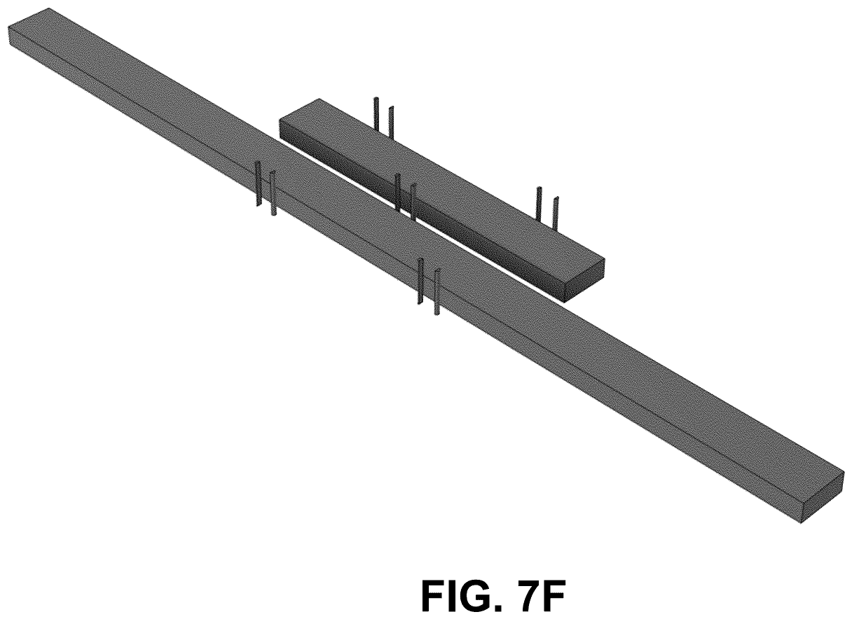

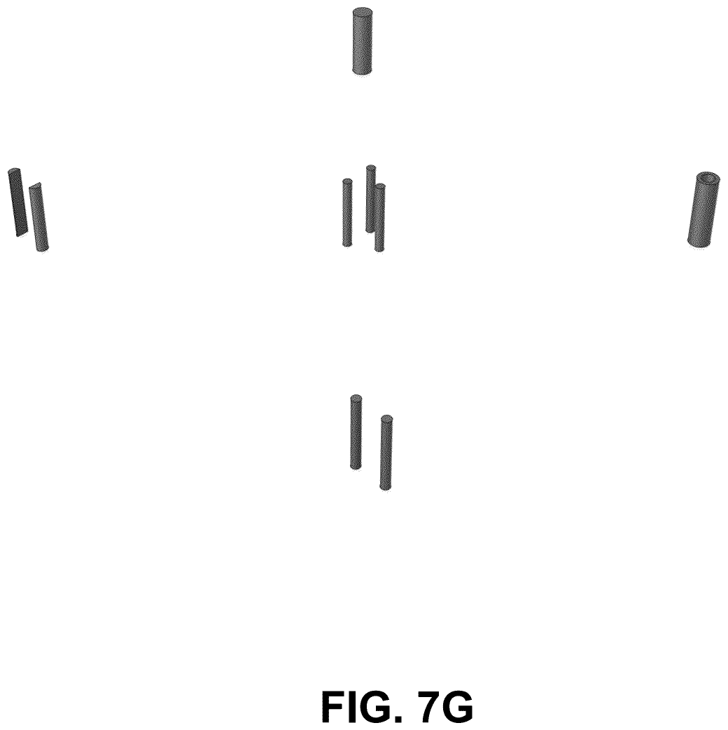

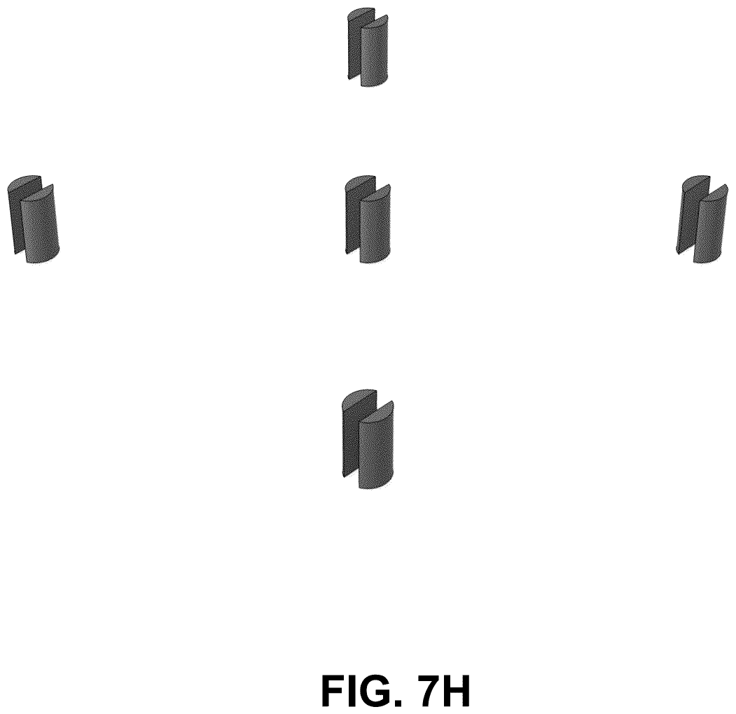

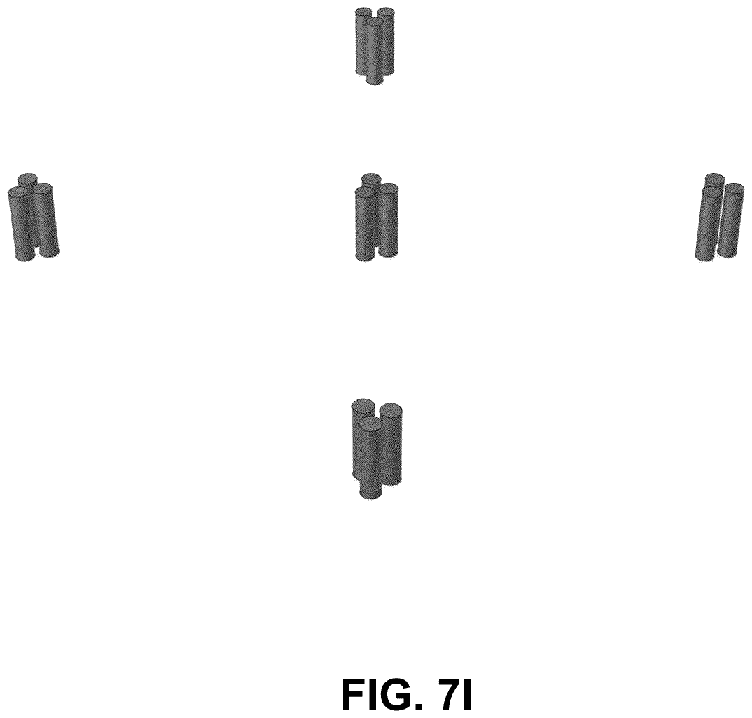

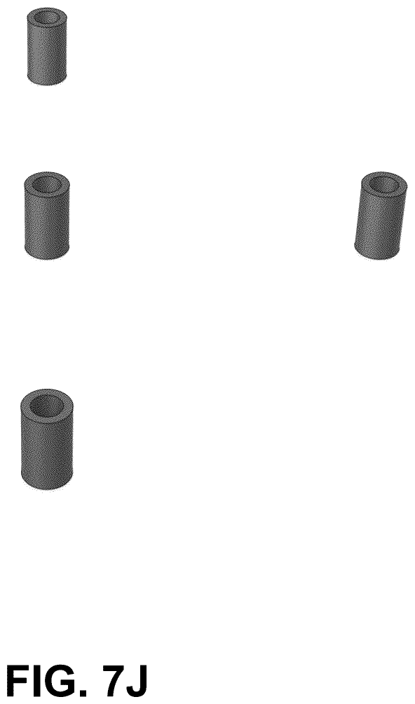

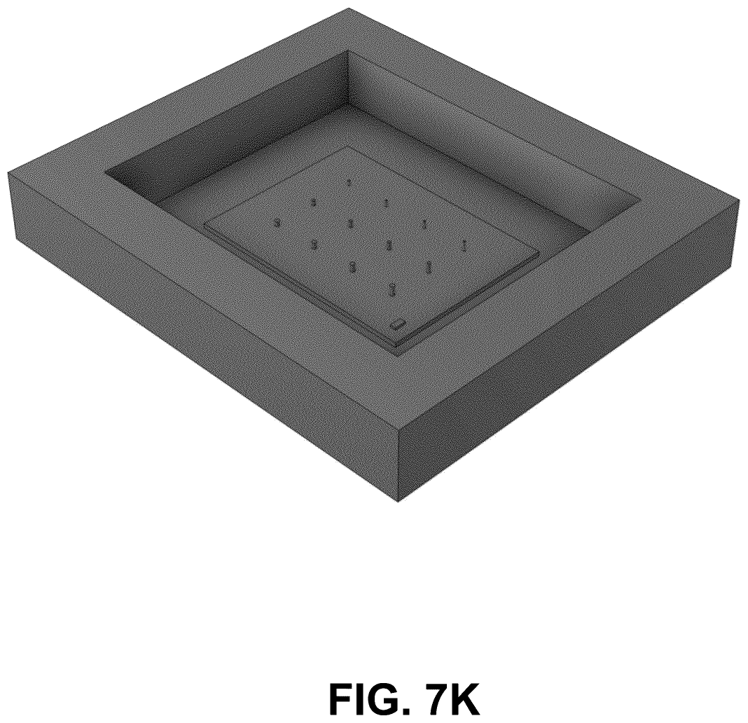

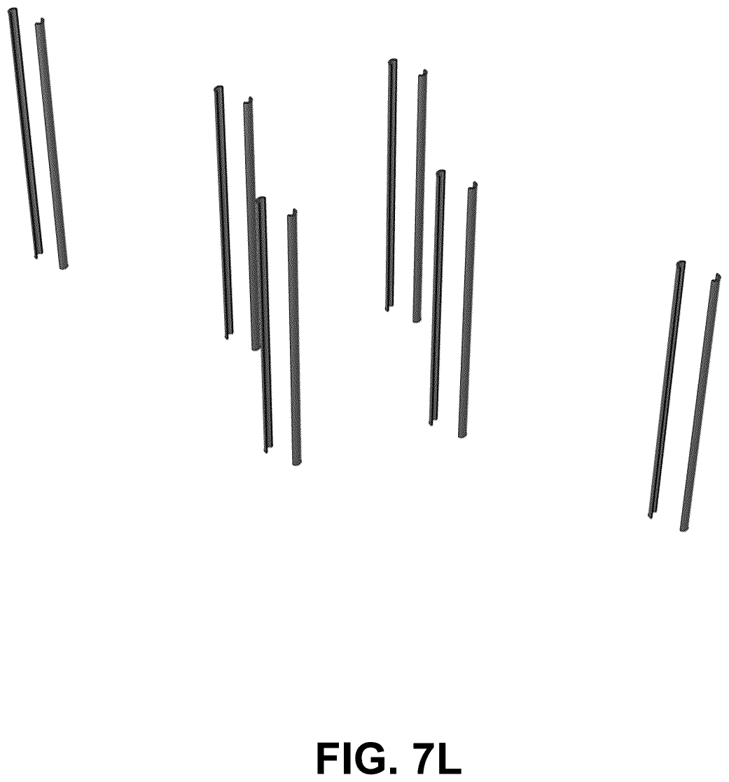

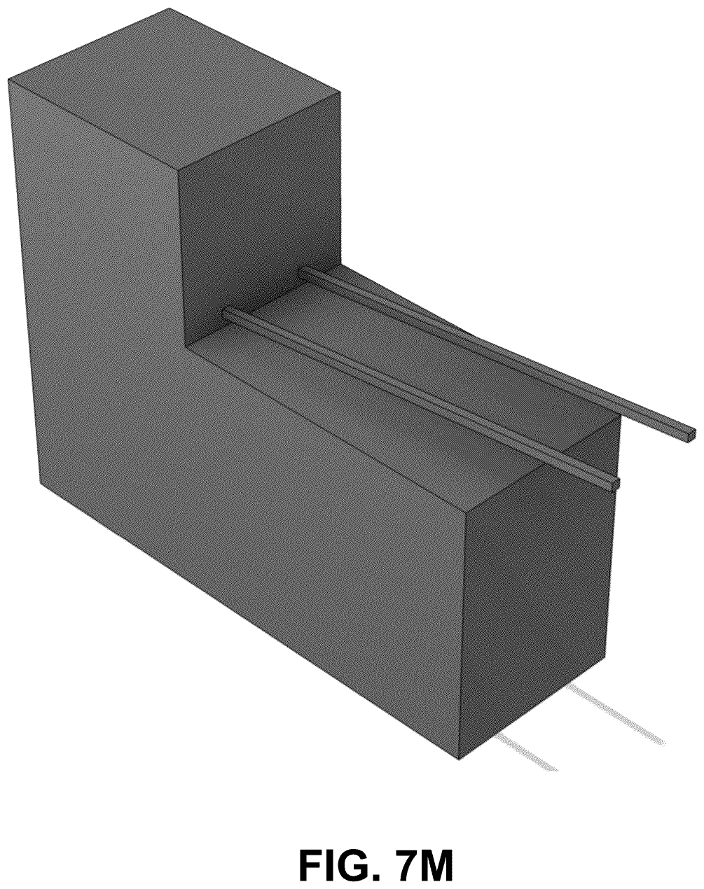

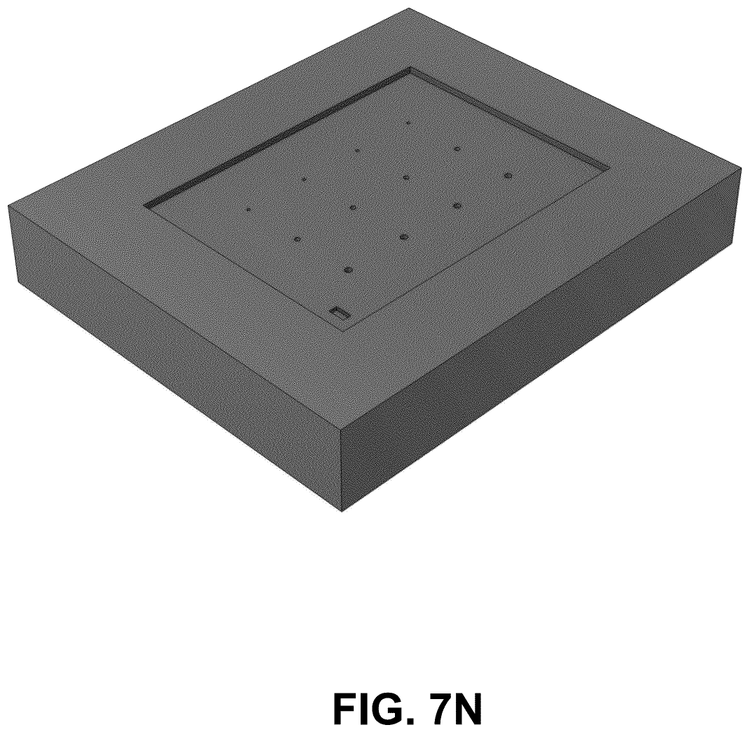

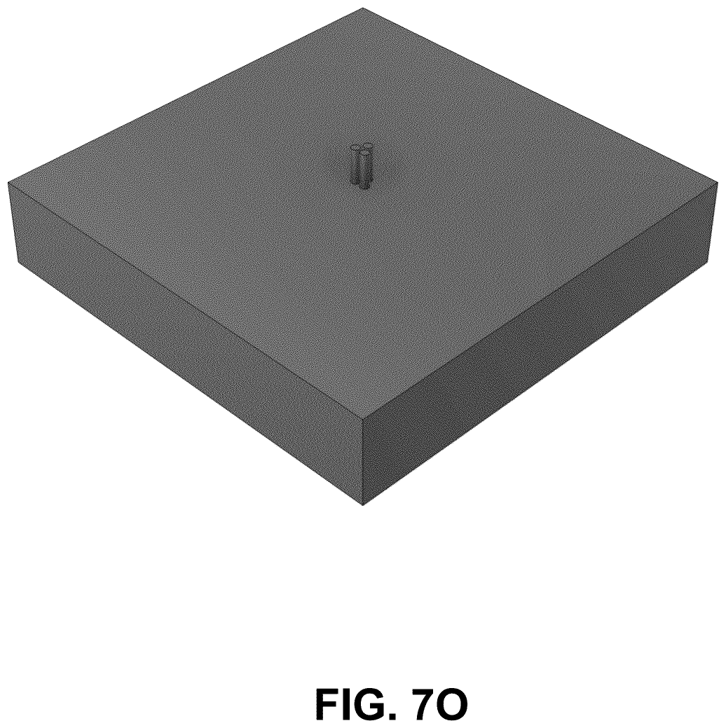

According to various embodiments, one or more micropillars can be printed onto a substrate. In various aspects, a micropillar can be a cylindrical structure or a semicylindrical structure. In some aspects, a micropillar can be a solid structure or can be a hollow structure. In some aspects, a micropillar can be a solid cylindrical structure (e.g., FIG. 7A), a hollow cylindrical structure (e.g., FIG. 7B), a “U” shaped solid structure (e.g., FIG. 7C), a solid or hollow semicylindrical structure (e.g., FIG. 7H and FIG. 7L, respectively), or a solid or hollow rectangular structure (e.g., FIG. 7M). In various aspects, a plurality of micropillars can be grouped together (e.g., pairs of two, groups of three, groups of any other number of micropillars).

According to various embodiments, fabrication of the micropillars can be based at least in part on light-based three-dimensional (3D) printing techniques, such as stereolithography or laser assisted printing. These light-based techniques can allow the use of a variety of chemistries for the polymer backbone, such as PEG, gelatin, collagen, hyaluronic acid, methocellulose, chitosan, 1,3-diglycerolate, silk fibroin, and polyvinyl alcohol. The polymers can then be functionalized with acrylate, methacrylate, norbornene, thiol, vinyl sulfone, and maleimide groups which can allow for use of free radical polymerization to form the pillars. This free radical polymerization can be initiated in the presence of UV light and a photoinitiation, such as lithium phenyl (2,4,6-trimethylbenzoyl) phosphinate (LAP), Irgacure 2959, Eosin Y, ruthenium/sodium per sulfate, 2,4,6-trimethylbenzoyl-diphenylphosphine oxide, and 2,2′-Azobis[2-methyl-N-(2-hydroxyethyl)propionamide], causing gelation of the hydrogel precures solution. In some examples, the polymer concentration can be lowered to achieve an effect on mechanical properties of the pillars (e.g., elasticity, hardness, plasticity, toughness, yield strength, tensile strength, stiffness, etc.). In some examples, other printing techniques can be used, such as two-photon polymerization, to maintain the high resolution and print fidelity of the pillars.

According to various embodiments, the pillar can be printed onto a substrate, such as glass, that is functionalized to allow for use for different applications and ease of handing. Functionalized glass bottom plates can allow for multiple prints in one container. In some examples, pillars can be fabricated onto functionalized coverslips that can allow them to be moved and manipulated for a broader range of applications.

In various embodiments of the present disclosure, digital light processing (DLP) printing can be used to fabricate the micropillars. In some examples, the micropillars can be fabricated using multiphoton lithography, stereolithography, masked stereolithography, liquid crystal display (LCD) 3D printing, and volumetric printing.

The present disclosure provides a device for quantifying biomechanical forces of a tissue including a plurality of micropillars configured to thread through the tissue. In various aspects, a device can include one or more blocks adjacent to the plurality of micropillars configured to elevate the tissue to increase a bending moment on each of the plurality of micropillars, wherein a displacement of each of the plurality of micropillars is based at least in part on the bending moment and a contractile force of the tissue.

In various aspects, each of the plurality of micropillars can have a length of from approximately 0.01 millimeters to approximately 20 millimeters. In some aspects, the length can be from approximately 0.05 millimeters to approximately 15 millimeters. In further aspects, the length can be from approximately 1 millimeter to approximately 10 millimeters.

In some aspects, each of the plurality of micropillars can have a semicircular or circular cross section with a radius of from approximately 0.0001 millimeters to approximately 5 millimeters. In some aspects, the radius can be from approximately 0.0005 millimeters to approximately 3 millimeters. In further aspects, the radius can be from approximately 0.001 millimeters to approximately 3 millimeters.

In some aspects, each of the plurality of micropillars can be approximately 0.05 millimeters to approximately 5 millimeters apart. In some aspects, each of the plurality of micropillars can be approximately 0.01 millimeters to approximately 3 millimeters apart. In further aspects, each of the plurality of micropillars can be approximately 0.1 millimeters to approximately 1 millimeter apart.

In some aspects, each of the one or more blocks can be approximately 0.001 millimeters to approximately 5 millimeters away from each micropillar. In some aspects, each of the one or more blocks can be approximately 0.005 millimeters to approximately 3 millimeters away from each micropillar. In further aspects, each of the one or more blocks can be approximately 0.01 millimeters to approximately 1 millimeter away from each of the plurality of micropillars.

In some aspect, the micropillars can include polyethylene glycol (PEG), gelatin, collagen, hyaluronic acid, methocellulose, chitosan, 1,3-diglycerolate, silk fibroin, polyvinyl alcohol, or a combination of any thereof. In some aspects, the micropillars can include an acrylate, a methacrylate, a norbornene, a thiol, a vinyl sulfone, a maleimide, or a combination of any thereof.

The present disclosure also provides a method of quantifying biomechanical forces of a tissue, including synthesizing a hydrogel bioink, fabricating one or more micropillars on a substrate, where the one or more micropillars include the hydrogel bioink, threading the one or more micropillars through an inner diameter of the tissue, measuring a displacement of each of the one or more micropillars over a period of time, and calculating a force generated by the tissue based at least in part on the displacement of each of the one or more micropillars.

In various aspects, the hydrogel bioink can include one or more polymers. In some aspects, one or more polymers can include polyethylene glycol (PEG), gelatin, collagen, hyaluronic acid, methocellulose, chitosan, 1,3-diglycerolate, silk fibroin, polyvinyl alcohol, or a combination of any thereof. In further aspects, one or more polymers can include an acrylate, a methacrylate, a norbornene, a thiol, a vinyl sulfone, a maleimide, or a combination of any thereof.

In various aspects, fabricating one or more micropillars on the substrate can be performed using a 3D printer. In some aspects, the 3D printer can be selected from a light-based 3D printer, a material extrusion printer, and an inkjet bioprinter. In some aspects, the light-based 3D printer can be a stereolithography 3D printer, a laser-assisted printer, a digital light processing printer, a multiphoton lithography printer, a masked stereolithography printer, a liquid crystal display (LCD) 3D printer, or a volumetric printer. In some aspects, the substrate can include a glass material.

The present disclosure can also provide a system for quantifying biomechanical forces of a tissue including one or more micropillars configured to thread through the tissue, a first device configured to measure a displacement of each of the one or more micropillars, where the displacement is based at least in part on a contractile force of the tissue, and a second device configured to calculate the contractile force generated by the tissue based at least in part on the displacement of each of the one or more micropillars.

In some aspects, the first device, the second device, or both can include a computing device comprising a processor and a memory, and machine-readable instructions. The machine-readable instructions, when executed by the processor, can cause the computing device to at least measure a displacement of each of the one or more micropillars, where the displacement is based at least in part on a contractile force of the tissue, and calculate the contractile force generated by the tissue based at least in part on the displacement of each of the one or more micropillars.

In some aspects, the system can further include one or more blocks adjacent to the one or more micropillars configured to elevate the tissue to increase a bending moment on each of the one or more micropillars, where the displacement of each of the one or more micropillars is based at least in part on the bending moment and the contractile force of the tissue.

### EXAMPLE 1

Various embodiments of the present disclosure can provide micropillar constructs for measuring contractile force of annular tissues. Quantifying the mechanical forces generated by cells and tissues with complex 3-dimensional geometries upon their substrates and environment is important for understanding many biological processes such as tissue morphogenesis, wound healing, and collective cell migration. Current methods for quantifying forces generated by tissues on their environment such as Traction Force Microscopy (TFM), support primarily planar tissue geometries. Existing elaborately fabricated constructs built to infer forces generated by natively contractile tissues may be repurposed for this use case, but they often either do not support tissues at the 300-1000 μm scale, or are too stiff to be sensitive to forces weaker than 10 μN. The present disclosure provides a platform to infer weak biological forces generated by contractile toroidal-shaped tissue explants through the fabrication of a novel micropillar construct. In addition, micropillars of the present disclosure can be conjugated with proteins or other active biomimetic peptides to investigate or modulate the force-generating properties of complex biological tissues, such as the natively contractile annular-shaped tissues used in demonstration.

### Methods

### Design

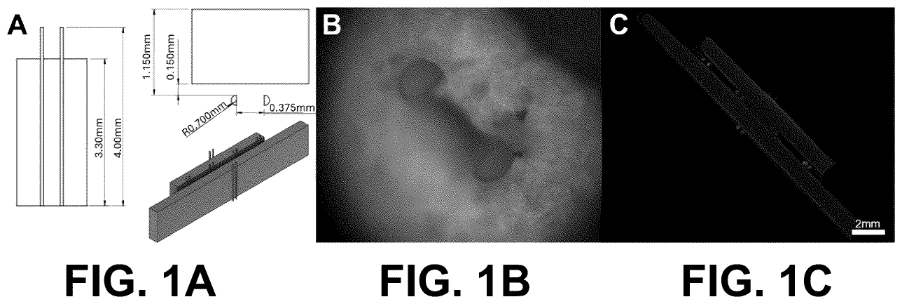

A novel micropillar construct was designed to quantify the forces generated by toroidal tissue geometries. Each selected pillar specification is 4,000 μm in height, with a semicircular cross-section 50 μm in radius. Each construct contains two pillars separated by 375 μm. Pillars are placed in close proximity to blocks printed with the same material. The blocks increase the sensitivity to weak contractile forces by elevating the tissue 1300 μm above the base of the pillars to increase the moment arm at which contractile forces can deflect the pillar structure as displayed in FIG. 1A. The final pillar dimensions can be small enough to be threaded through toroidal shaped explant tissue with an inner diameter of 475 μm (FIG. 1B).

### Fabrication

While the methods and systems provided herein are flexible and can be realized with varying materials and printer technologies, the pillars used as an example here were fabricated using a digital light processing (DLP) bioprinter and a hydrogel bioink. DLP printer bioink was formulated using polyethylene glycol diacrylate (PEGDA, 12.5 v %, 200 Da), four-arm polyethylene glycol acrylate (PEG-Ac, 5 w/v %, 10 kDa), and a solution of 20 mM lithium phenyl-2,4,6-trimethylbenzoylphosphinate (LAP) photoinitiator for use with the DLP printer. Designs were printed using 50 μm vertical slices at 30% light intensity at 0.02 mm/s printer probe velocity.

Glass bottom plates were acrylated to allow the structure to adhere to them during the printing process. Pillar designs were printed in standard 6 well glass bottom black plates with a 1.5 cover glass. The plates were plasma cleaned with air for two minutes then a drop of 3-(Trimethoxysilyl)propyl acrylate was added to coat the bottom of each well. The plate is then placed under vacuum to dry for 60 hours covered from light. The plate was then stored at 4° C. till used.

For characterizing the mechanics of the printed structures as described in the section titled Material Characterization, the structures were printed on 22 mm glass coverslips acrylated via treatment with the same methods described for preparing the glass bottom plates above.

### Example Tissue Preparation

Toroidal tissue preparations were explanted through microsurgery of stage 11.5 Xenopus laevis embryos. Briefly, the vitelline membrane of the embryo was removed and a superficial incision was created along the equator of the spherical embryo. The epithelial tissue “cap” of the embryo superior to the incision was lifted without damaging the underlying mass of tissue underneath. A toroidal tissue structure was then created by bisecting the embryo at the initial equatorial incision to isolate a mantle of tissue that is revealed by lifting the epithelial cap from the developing embryo. This yielded a toroidal shaped tissue explant that contracts natively.

### Experimental Application

Toroidal explants of contractile tissue were prepared from Xenopus laevis embryos at stage 11.5 as described in subsection Example Tissue Preparation, and the micropillar structure was threaded through the explant of initial inner diameter of approximately 500 μm. The pillar structures were imaged continuously for 60 minutes using fluorescent stereo zoom microscopy (FIG. 1B). To infer forces generated by the explant, a constitutive relation of exerted force and beam end deflection were derived taking into account pillar material stiffness and pillar geometry.

### Material Characterization

### (i) Dynamic Mechanical Analysis

Rheological behavior of bulk PEG ink gel was characterized using a rheometer (DHR3, TA instruments), equipped with an 8 mm parallel plate geometry. An 8 mm Poly(dimethyl)siloxane Sylgard 184 kit (Dow Corning) mold was used to fabricate a bulk gel. The gel was allowed to swell overnight in PBS at room temperature. Dynamical mechanical analysis (DMA) in compression was done at 5 μm/s until failure. DMA in tension was done at the same rate until failure, but the rectangle tension fixture was used and the PEG ink was cured to form a slab that is 9 mm long.

### (ii) Printed Structure Deflection

The force-displacement relationship of the fabricated structures was determined using a micro-scale tension-compression device (MicroSquisher, CellScale). During testing, a cylindrical probe 100 μm in diameter was used to horizontally deflect printed pillar structures in a cyclic loading-unloading pattern. During loading cycles, the testing probe applied forces of 0.5, 1, 2, and 4 μN of force from which resulting pillar displacement was measured.

### Material Patterning

After printing, a 1 mM thiolated rhodamine B (GCDDD-rhodamineB) and 1 mM LAP solution was added to the pillars and let sit at room temperature for 45 minutes to diffuse into the hydrogel pillars. After which each well was placed under UV light at 15 mW/cm\hat{ }2 (Omnicure 320-390) for 60 s. The thiolated rhodamine B and LAP solution was removed, and PBS was added to the pillars. The pillars sat at 4° C. overnight to allow unreacted thiolated rhodamine B to diffuse out before imaging.

### Results

Toroidal tissue explants were prepared and placed upon fabricated micropillar constructs as shown in FIG. 1B to demonstrate the application of the present disclosure. Printed pillar structures were small enough to be threaded through biological tissue preparations to observe the active contraction of the tissue upon the structure.

### Reference

Davidson, Lance A., et al. “Mesendoderm Extension and Mantle Closure in Xenopus laevis Gastrulation: Combined Roles for Integrin Alpha(5)Beta(1), Fibronectin, and Tissue Geometry.” Developmental Biology, vol. 242, no. 2, February 2002, pp. 109-29. PubMed, https://doi.org/10.1006/dbio.2002.0537.

### EXAMPLE 2

During development, cells and tissues move and often extend large distances where they ultimately settle into anatomical structures representative of each stage of embryonic development. These tissues deform significantly in three dimensions in vivo due to biomechanical forces generated natively within the tissue. Resolving the magnitude of these biomechanical forces can be useful for investigating the underlying biological mechanisms from whence these forces are derived. Because multiple tissue movements occur at once during the morphogenesis of the Xenopus laevis gastrula, resolving the relative magnitudes of force-generating events can allow for understanding the relative roles of these mechanisms towards the proper development of the embryo.

The action of tissue surface tension is one such effector of cell migration that contributes to the large tissue deformations and tissue rearrangements observed during gastrulation. Tissue surface tension results in tissue self-organization in many tissues throughout the embryo. The surface tension of the different germ layers during gastrulation has been observed to result in their stratification and migration during the development of the zebrafish and the amphibian Rania pipiens. During gastrulation of the Xenopus laevis embryo, surface tension has been implicated in multiple cell migratory movements. Surface tension has been reported to be involved in radial intercalation events that allow for spreading of mesendoderm during migration and eventual closure of the mesendoderm mantle, force-generating convergent extension events that contribute to tissue movements originating in the involuting marginal zone of tissue and convergent thickening, an additional convergence force that contributes to closure of the blastopore at the vegetal pore of the embryo.

The Differential Adhesion Hypothesis (DAH) posited by Malcom Steinberg explains the fluidlike behavior of embryonic tissues spreading upon one another. These include the wetting or spreading of cells and tissues upon their substrates, the sorting of different populations of embryonic cells in vivo, and the aggregation of cells into tissues. The DAH describes these behaviors as resulting from differences in tension at the interfacial plane of one cell type to another, due to varying concentrations of cell adhesion molecules in different cell and tissue types, which results in varying adhesivity at the interface of one tissue type to another. In addition to differential adhesion, tissue surface tension has been observed to be affected by active actomyosin mediated contractility at the cellular cytoskeleton which, across many cells, affects the surface tension of cellular aggregates and large tissues.

Quantifying the force generated by surface tension mediated processes during development is challenging. Surface tension mediated processes appear to generate forces on the scale of micronewtons, requiring sensitive experimental setups to measure. In the Xenopus laevis embryo, Shook et. al, 2018 (Shook et al., 2018) quantified tensile convergence forces at the marginal zone in the range of 1.5-4 μN using a tractor pull assay designed to measure these forces of the involuting marginal zone tissue explants. Feroze et. al, 2015 (Feroze et al., 2015) measured the force of blastopore lip closure at approximately 0.5 μN using calibrated cantilevers inserted into the margins of the closing blastopore. While these processes occur in tissue on the opposite end of the embryo to the migrating mesendoderm mantle at the animal pole, because they are surface tension driven processes, closure of a toroidal explant in the absence of cellular crawling can be due to forces of similar magnitude. Thus, to quantify the surface tension driven process of closure of a toroidal (or donut) shaped explant of the migratory mesendoderm mantle, a contractility of <10 μN in magnitude can be measured.

Open source and commercial solutions exist to quantify forces of cells and tissues upon their substrate and each other. These include solutions such as microneedle arrays or traction force microscopy to quantify traction forces of cellular crawling for collectively migrating mesendoderm or millipillar constructs built for assessing engineered cardiac tissues. These constructs can be insufficient because they either accommodate mainly planar tissue geometries rather than the toroidal tissue geometry representative of the in vivo mesendoderm, or they have been described to measure millinewton forces as in the case of the millipillars and are often not sensitive to the micronewton scale of force expected from the explant. The individual assays previously cited used by Shook and Feroze were successful in part because they were developed specifically to accommodate the explant. The present disclosure develops a flexible platform that can be used to directly measure or infer the force of a natively contractile toroidal explant. In various examples, the construct can accommodate the toroidal tissue geometry with an inner diameter of approximately 500 μm.

Digital light processing (DLP) based 3D bioprinting technology presents a promising method for fabricating a measurement platform to accommodate the tissue explant because of its demonstrated uses in fabricating high-resolution, geometrically elaborate designs out of polymerized hydrogel. Using DLP based bioprinting, a design can be specified using computer-aided design (CAD) software that can accommodate the representative in vivo geometry of the toroidal explant and tune bioink composition to allow for a soft enough printed hydrogel material to be sensitive to measure the toroid's native contractility. In various examples, the present disclosure can be used to design, print, and apply a DLP printed hydrogel load cell for measuring the native contractility of the toroid explant.

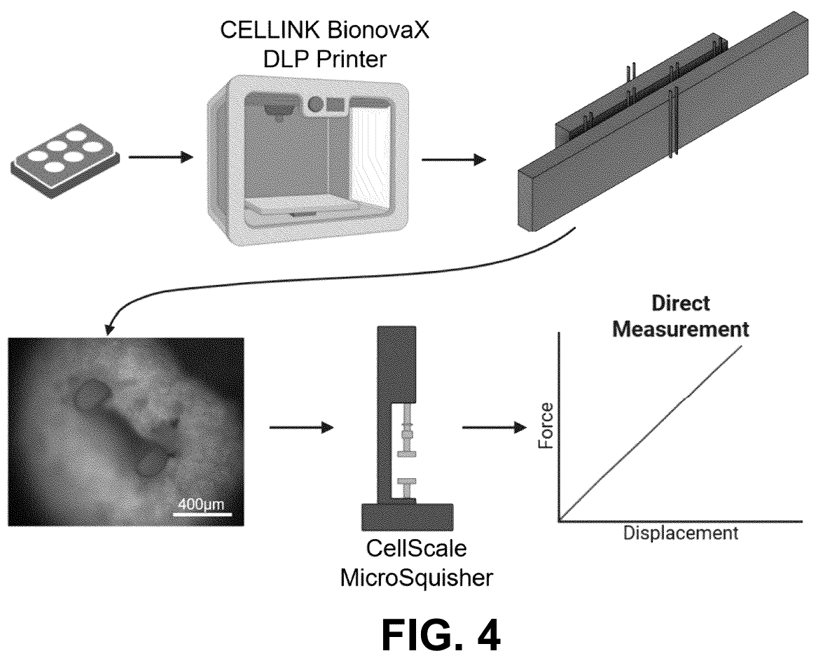

Measuring the toroid explant presents multiple challenges. The explant generates weak contractile forces that can be <10 μN natively, the toroid is approximately 0.5 mm tall and 1 mm in diameter, and toroid explants often remain submerged in media and out of contact with an air water interface. To this end, a micropillar construct was designed to be printed in a standard 6-well glass-bottom plate using a commercial CELLINK BionovaX DLP printer. The designs print on circular microscope coverslips in order to facilitate direct measurement with a mechanical testing device. FIG. 4 demonstrates the fabrication process, whereby standard 6-well plates are acrylated to allow polymerized hydrogel to adhere to their surface when printed with the DLP printer. Designs are printed directly into the wells or onto coverslips, leaving enough room in each well for the explantation of tissue and positioning on the printed pillars. The pillar design is then imaged under a Zeiss AxioZoom fluorescence stereo microscope to observe and record explant behavior and any pillar deflection. Pillar designs can then be directly measured using a micromechanical testing device (CellScale MicroSquisher) and the force generated by the explant can be inferred from the force-displacement relationship of the printed construct derived from micromechanical testing of the construct in isolation.

To quantify the forces generated by toroidal tissue geometries and leave enough working room within the well of a 6-well plate for tissue manipulation, the design detailed in FIG. 1A was used. Each selected pillar specification is 4,000 μm in height, with a semicircular cross-section 70 μm in radius. Each construct contains six sets of two pillars, where each pillar in a pair is separated by 375 μm to accommodate the approximately 475 μm inner radius of a toroidal explant. Pillars are placed in close proximity to blocks printed with the same material. The blocks increase the sensitivity of the design to detect weak contractile forces by elevating the tissue 1300 μm above the base of the pillars to increase the moment arm at which contractile forces may deflect the pillar structure. The final pillar dimensions can be small enough to be threaded through toroidal shaped explant tissue with an inner diameter of approximately 475 μm.

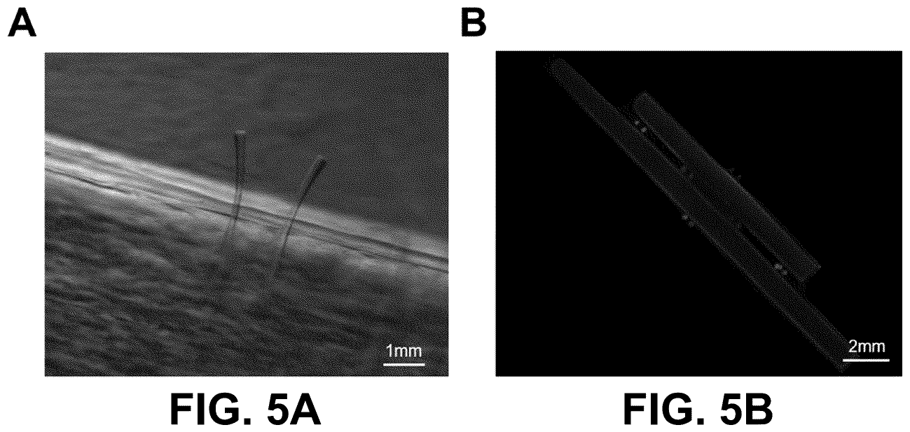

Pillars were printed using a bioink that included polyethylene-glycol diacrylate (PEG-DA) and four-arm polyethylene glycol acrylate (four-arm PEG-Ac). This mixture was chosen to obtain a pillar material soft enough to deflect, given weak biomechanical forces, yet stiff enough to retain its structure given its high aspect ratio of 4 mm tall and 70 μm in cross-section diameter. PEG-Ac softens the material after polymerization. Rhodamine B fluorescent dye was also added to the bioink to label the pillars and view under the fluorescence microscope. Labeled pillars with a positioned biological explant are displayed in FIG. 4. Printed designs were incubated with thiolated rhodamine-B and imaged as shown in FIG. 5B. This indicates that these prints can be patterned with bioactive peptides for the modulation of biological processes of interest. This modulation can potentially assist in the investigation of the biological mechanism to which biomechanical forces are generated and modulated in vivo. Representative prints are displayed in FIG. 5, where FIG. 5B displays a design incubated with thiolated rhodamine-B.

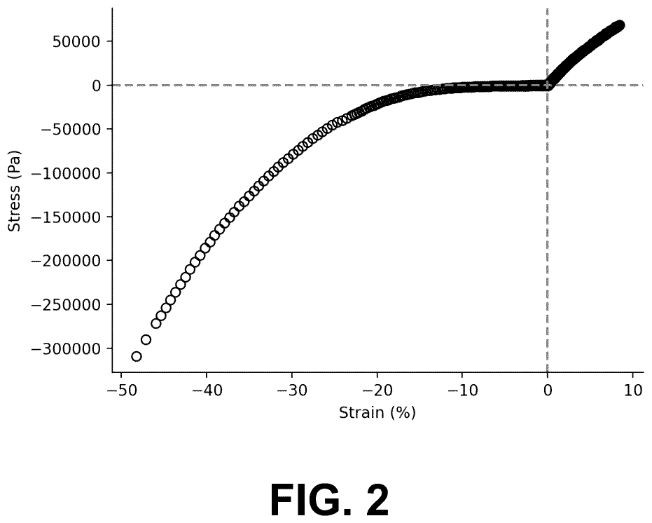

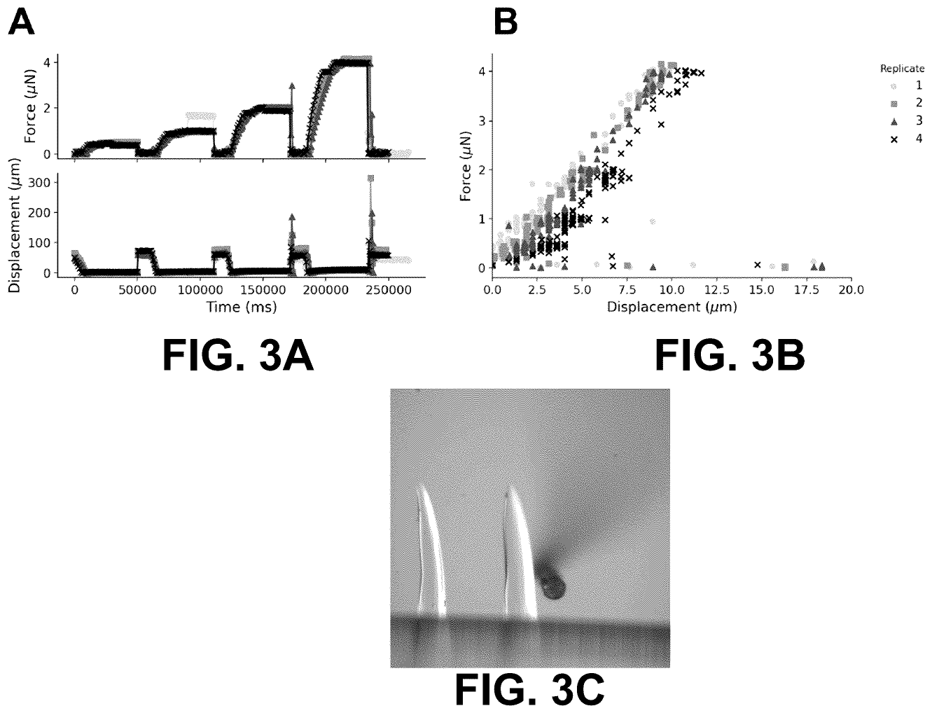

To infer forces applied by explants on the pillar constructs, a CellScale MicroSquisher was used to perform cyclic loading (FIG. 3A) of progressively greater horizontal forces to deflect the pillars in an experimental setup (FIG. 3C). Micromechanical testing revealed linear force-displacement curves in loading and unloading for multiple replicates (FIG. 3B). To further characterize and understand the material response under mechanical loads, dynamic mechanical testing was performed to derive a stress-strain relationship of the material. The stress-strain relationship measured from tests in uniaxial tension and compression were displayed (FIG. 2) and it was found that the material is softer in compression than in tension. The hydrogel material appears brittle in tension. During bending of a pillar structure, the material would be expected to be experiencing both tension and compression stresses.

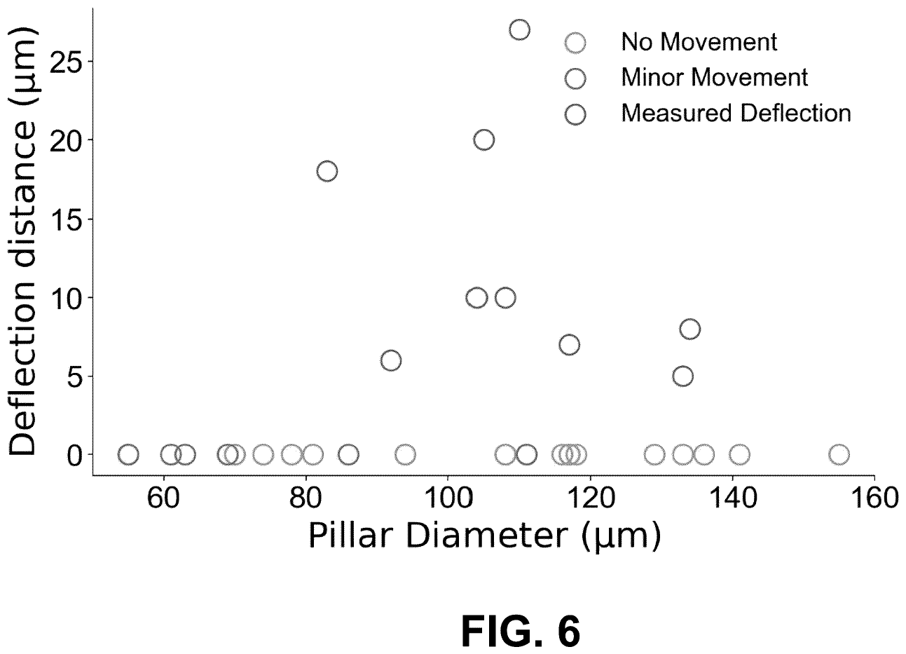

Experimental tests of a previous pillar design including 2 mm tall pairs of pillars at the same diameter are shown in FIG. 6. This data includes 16 experimental yielding deflection data from 34 pillars (16 pairs of pillars). Unfortunately, measured pillar diameters after fabrication were highly variable. Measured pillar deflections for different pillar replicates of this design were also variable. Some designs displayed a minor amount of oscillating movement in response to biomechanical forces generated by the contractile explant; however, these movements were minimal such that determining a specific measurement for deflection from microscopy was difficult. These replicates are labeled as “Minor movement” in FIG. 6.

The present disclosure defines a hydrogel construct that can be fabricated with a DLP printer and characterizes the mechanical properties of the fabricated construct. Use of the construct to infer the magnitude of force generated by a natively contractile toroidal tissues extirpated from Xenopus laevis gastrulae was demonstrated. Experimental measurements were performed with 2 mm tall pillars which were insensitive to the forces generated by multiple toroidal explants. This motivated the design printed, patterned, and displayed in FIG. 5. Increasing the height of the overall structure can further increase the moment arm at which biomechanical forces act to bend the pillar construct. This can allow the structure to be more sensitive to weaker forces.

Tissue surface tension is thought to be a function of both cortical tension and intercellular adhesion. Alterations in cytoskeletal acto-myosin activity (affecting cortical tension) or intracellular signaling cascades that affect expression of cell adhesion molecules (resulting in differential intercellular adhesion) might alter and help explain the native physiological behavior of tissues in the context of their in-vivo environment. An applicable example of this is the observation that Lewis lung carcinoma cells demonstrate increased propensity for invasion associated with loss of E-cadherin. It has previously been observed that invasive activity can be suppressed through increasing expression of either P or E-cadherin. It has also been found that treatment with the common corticosteroid dexamethasone increased expression of cadherin and decreased invasiveness of human fibrosarcoma cells in culture. An assay sensitive to decreases in contractility due to perturbations in the mechanisms of tissue surface tension can therefore be useful to identify and investigate additional effectors of tissue surface tension.

The explants in the present experiment produced enough biomechanical force to deflect the fabricated pillars. The force-displacement relationship derived through micromechanical testing implies that any observed movement of the pillars likely is the result of forces on the scale of micronewtons. Previously, the major driving force for mesendoderm mantle closure at the animal pole of the Xenopus laevis embryo at this stage of gastrulation was thought to be due to lamellipodial protrusive behavior of migratory cells at the leading-edge of the tissue. However, multiple studies quantifying the forces generated through lamellipodial extension suggest that the magnitude of these forces are in the range of 0-30 piconewtons. Additionally, traction force microscopy experiments performed previously on the migratory behavior of the mesendoderm tissue at this stage of development reveal average traction stresses of approximately 200 Pa at the leading edge, which resolves to 0.012 μN of force when considering the 8 μm×8 μm areas over which stresses were calculated. These findings suggest that the native contractility of the explant, thought to be due to tissue surface tension, results in forces of at least 2-orders of magnitude greater than that of crawling. Therefore, tissue surface tension is either a major driving mechanism of mesendoderm mantle closure, or is modulated in vivo during the native development of the embryo.

Finally, the ability to pattern these hydrogel constructs with thiolated bioactive peptides was also demonstrated. This can allow for investigation of the modulation of the mechanisms responsible for the generation of biomechanical forces measured by the construct. The native tissue surface tension properties of the explanted tissue can drive observed tissue reorganization behavior. The magnitudes of these forces can be orders of magnitude greater than cell crawling, which was previously understood as a primary mode of collective migration of this tissue in vivo.

The present disclosure provides a hydrogel construct used to infer the relative magnitude of a natively contractile mesendoderm explant that represents the native geometry of the tissue in vivo. In addition, the potential for the hydrogel explant to be patterned with bioactive peptides to assist in further investigation of factors that may modulate biomechanical forces has been demonstrated. While the proposed geometry can be specific to the tissue explant, the material characterization of the hydrogel material described can be applicable regardless of construct geometry. Additionally, using a hydrogel material polymerized through DLP technology can allow for specification of arbitrary geometry that can be customized to be well suited to other experimental setups.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs.

Those skilled in the art will recognize or be able to ascertain using no more than routine experimentation, many equivalents to the specific embodiments of the invention described herein. Such equivalents are intended to be encompassed by the following claims.

What is claimed is:

1. A device for quantifying biomechanical forces of a tissue, comprising:
a plurality of micropillars configured to thread through the tissue; and
one or more blocks adjacent to the plurality of micropillars configured to elevate the tissue to increase a bending moment on each of the plurality of micropillars, wherein a displacement of each of the plurality of micropillars is based at least in part on the bending moment and a contractile force of the tissue.

2. The device of claim 1, wherein each of the plurality of micropillars has a length of from approximately 1 millimeter to approximately 10 millimeters.

3. The device of claim 1, wherein each of the plurality of micropillars has a semicircular cross section with a radius of approximately 0.001 millimeters to approximately 3 millimeters.

4. The device of claim 1, wherein each of the plurality of micropillars is approximately 0.01 millimeters to approximately 1 millimeter apart.

5. The device of claim 1, wherein each of the one or more blocks is approximately 0.1 millimeters to approximately 1 millimeter away from each of the plurality of micropillars.

6. The device of claim 1, wherein the plurality of micropillars comprise polyethylene glycol (PEG), gelatin, collagen, hyaluronic acid, methocellulose, chitosan, 1,3-diglycerolate, silk fibroin, polyvinyl alcohol, or a combination of any thereof.

7. The device of claim 1, wherein the plurality of micropillars comprise an acrylate, a methacrylate, a norbornene, a thiol, a vinyl sulfone, a maleimide, or a combination of any thereof.

8. A method of quantifying biomechanical forces of a tissue, comprising:
synthesizing a hydrogel bioink;
fabricating one or more micropillars on a substrate, wherein the one or more micropillars comprise the hydrogel bioink;
threading the one or more micropillars through an inner diameter of the tissue;
measuring a displacement of each of the one or more micropillars over a period of time; and
calculating a force generated by the tissue based at least in part on the displacement of each of the one or more micropillars.

9. The method of claim 8, wherein fabricating one or more micropillars on the substrate is performed using a three dimensional (3D) printer.

10. The method of claim 9, wherein the 3D printer is selected from a light-based three dimensional (3D) printer, a material extrusion printer, and an inkjet bioprinter.

11. The method of claim 10, wherein the light-based 3D printer comprises a stereolithography 3D printer, a laser-assisted printer, a digital light processing printer, a multiphoton lithography printer, a masked stereolithography printer, a liquid crystal display (LCD) 3D printer, a volumetric printer, or a combination of any thereof.

12. The method of claim 8, wherein the hydrogel bioink comprises one or more polymers.

13. The method of claim 12, wherein the one or more polymers comprise polyethylene glycol (PEG), gelatin, collagen, hyaluronic acid, methocellulose, chitosan, 1,3-diglycerolate, silk fibroin, polyvinyl alcohol, an acrylate, a methacrylate, a norbornene, a thiol, a vinyl sulfone, a maleimide, or a combination of any thereof.

14. A system for quantifying biomechanical forces of a tissue, comprising:
one or more micropillars configured to thread through the tissue;
a first device configured to measure a displacement of each of the one or more micropillars, wherein the displacement is based at least in part on a contractile force of the tissue; and
a second device configured to calculate the contractile force generated by the tissue based at least in part on the displacement of each of the one or more micropillars.

15. The system of claim 14, wherein each of the one or more micropillars has a length of from approximately 1 millimeter to approximately 10 millimeters.

16. The system of claim 14, wherein each of the one or more micropillars has a semicircular cross section with a radius of approximately 0.001 millimeters to approximately 3 millimeters.

17. The system of claim 14, wherein each of the one or more micropillars is approximately 0.1 millimeters to approximately 1 millimeter apart.

18. The system of claim 14, wherein the one or more micropillars comprise polyethylene glycol (PEG), gelatin, collagen, hyaluronic acid, methocellulose, chitosan, 1,3-diglycerolate, silk fibroin, polyvinyl alcohol, or a combination of any thereof.

19. The system of claim 14, wherein the one or more micropillars comprise an acrylate, a methacrylate, a norbornene, a thiol, a vinyl sulfone, a maleimide, or a combination of any thereof.

20. The system of claim 14, further comprising one or more blocks adjacent to the one or more micropillars configured to elevate the tissue to increase a bending moment on each of the one or more micropillars, wherein the displacement of each of the one or more micropillars is based at least in part on the bending moment and the contractile force of the tissue.
